(19) United States
(12) Patent Application Publication
(10) Pub. No.: US 2015/0226735 A1
(43) Pub. Date: Aug. 13, 2015

(54) SYSTEMS AND METHODS FOR ENZYME DETECTION

(71) Applicant: UNIVERSAL BIOSENSORS PTY LTD, Victoria (AU)

(72) Inventor: Alastair M. Hodges, Blackburn South (AU)

(21) Appl. No.: 14/429,744

(22) PCT Filed: Sep. 19, 2013
(86) PCT No.: PCT/IB2013/002582

§ 371 Date: Mar. 19, 2015

Related U.S. Application Data
(60) Provisional application No. 61/703,182, filed on Sep. 19, 2012.

(51) Int. Cl. G01N 33/543; G01N 27/327

(52) U.S. Cl. CPC G01N 33/54373; G01N 27/327

(57) ABSTRACT

Disclosed herein are a biosensor and a system including a biosensor and a meter for detecting a target analyte in a liquid sample via a cleavage reaction specific to the target analyte. Also disclosed are a method of fabricating the biosensor and a method of using the biosensor or the system.

### BACKGROUND

It is often important to be able to detect the presence or activity of molecules having enzymatic action. For example, enzymes whose mode of action is to cleave a bond are important in oncology and in such applications as monitoring liver function. At present these enzymes can be detected using traditional techniques such as immunoassays using wet reagents in a manual test or run on a large analyser. There is a need for rapid detection of such enzymes at the point of care so that timely action can be taken and the costs of, for example, an additional visit to draw blood, transportation of the samples to a laboratory, and/or reporting back of results to the physician can be avoided. To be practical for use as a point-of-care device, a test should be simple to use such that people with minimal training can successfully run the test, and it should be relatively fast, so that the result can be provided in a timely manner and the appropriate actions taken.

### SUMMARY

Some embodiments of the invention include a biosensor for detecting a target analyte in a liquid sample. The biosensor can include at least a first chamber and a second chamber, wherein the first chamber and the second chamber can be in fluid communication, wherein the first chamber can include a probe species, wherein the probe species can be retained in the first chamber by a linker, wherein the target analyte can cleave the linker to liberate the probe species into the liquid sample in the first chamber, wherein the biosensor can be configured to move the liquid sample from the first chamber to the second chamber, and wherein the liberated probe species can be detected in the second chamber via a detection mechanism. The linker can be attached to (e.g., absorbed to, tethered to, supported on, or the like) an internal surface of the first chamber. The linker can be attached to a separate support. For example, the separate support can include a bead. In some embodiments, the bead can be magnetic. The separate support can be immobilized or retarded in the first chamber. For example, the linker can be attached (e.g., to an internal surface of the first chamber or to a separate support) via a covalent bond or a non-covalent bond. In some embodiments, the non-covalent bond via which the linker is attached in the first chamber can include at least one bond selected from the group consisting of, for example, a streptavidin/biotin bond, a thiol/gold bond, and the like. The target analyte can include, for example, an enzyme. Thus, in some embodiments, the target analyte can include at least one enzyme selected from the group consisting of, for example, a chymotrypsin, a pepsin, a papain, an isopeptidase, a thrombin, a lactase, a maltase, a sucrase, an amylase, a pappalysin-2, a lysozyme, a protease, a matrix metalloproteinase, and the like.

Cleaving the linker to liberate the probe species can be specific to the target analyte. In some embodiments, the probe species can be linked to the linker via a bond a covalent bond, a non-covalent bond, or the like. The non-covalent bond can include at least one bond selected from the group consisting of, for example, a hydrogen bond, an electrostatic bond, and the like. In some embodiments, the target analyte can cleave the linker. The probe species can include, for example, an optically active molecule, an enzyme, an electrically active molecule, or the like.

In some embodiments, the liberated probe species can be detected directly via the detection mechanism. In some embodiments, the liberated probe species can undergo a detection reaction, wherein the detection reaction can generate a reaction product, wherein the reaction product can be detected via the detection mechanism. In some embodiments, the detection reaction can include at least one intermediate reaction and/or can generate at least one intermediate product. The detection chamber can include at least one reagent, wherein the at least one reagent can participate in the detection reaction (and/or at least one intermediate reaction if applicable). The reagent can include at least one reagent selected from the group consisting of, for example, a substrate, a mediator, a cofactor, a buffer, an electrochemical species, and the like. The substrate can include, for example, an enzyme substrate. The detection mechanism can include one mechanism selected from the group consisting of, for example, reflectance spectroscopy, transmission spectroscopy, fluorometry, turbidimetry, chemiluminescence microscopy, coulometry, amperometry, potentiometry, and the like.

The first chamber can include a reaction chamber, and wherein the second chamber can include a detection chamber. The biosensor can further include a filling chamber, wherein the filling chamber can be in fluid communication with the first chamber. The filling chamber can be proximal to the first chamber. The biosensor can be configured to move the liquid sample via capillary action. In some embodiments, the biosensor can be configured to move the liquid sample from the first chamber to the second chamber upon activation. For example, the first chamber can have a first height, wherein the second chamber can have a second height, wherein the second height can be smaller than the first height, and wherein the activation can include opening a vent in the second chamber. The vent can be located at the distal end of the detection chamber. The first chamber and the second chamber can also have the same or similar height and where the filling of the second chamber does not need to empty the first chamber of liquid. The second chamber can include two or more electrodes. Each of at least two of the two or more electrodes can be electrically connected to contact pads.

Some embodiments of the invention include a system for detecting a target analyte in a liquid sample, wherein the system can include a biosensor described herein and a meter. The system can further include a temperature control apparatus. In some embodiments, the temperature control apparatus can include a heater. In some embodiments, the system can further include a temperature measurement apparatus. The system can further include a temperature signalling apparatus to signal the temperature within the system. The temperature signalling apparatus can generate a signal when the temperature within the system is suitable for detecting the target analyte. The temperature signalling apparatus can generate a signal when the temperature within the system is not suitable for detecting the target analyte. The signal can include, for example, an audible signal, a visual signal, or the like. The meter can be reusable. The biosensor of the system can include two or more electrodes, wherein each of at least two of the two or more electrodes can be electrically connected to a contact pad, wherein the contact pads can be electrically connected to the meter. In some embodiments, the system can generate a stimulation for the detection. The stimulation can include at least one stimulation selected from the group consisting of, for example, an electrical stimulation, an optical stimulation, and the like. The electrical stimulation can include at least one stimulation selected from the group consisting of, for example, a current, a potential, and the like. The optical stimulation can include, for example, a light including one or more wavelengths. In some embodiments, the stimulation can be constant. In some embodiments, the stimulation can vary with time. In some embodiments, the system can include a timing mechanism. The system can include a mechanism to activate the advance of the liquid sample from the first chamber to the second chamber of the biosensor. The system can further include a mechanism to generate a result in a desired format. The system can further include a mechanism to convey the result in the desired format. The mechanism to convey the result can include, for example, a screen, a speaker, a printer, or the like.

Some embodiments of the invention include a method of detecting a target analyte in a liquid sample using a biosensor and/or a system disclosed herein. The system can include a biosensor and a meter. The biosensor can include a first chamber and a second chamber. The first chamber can include a probe species, wherein the probe species can be retained in the first chamber via a linker, and wherein the target analyte can cleave the linker to liberate the probe species. The method can include providing the liquid sample; allowing the liquid sample to remain in the first chamber of the biosensor to generate a reacted liquid sample; advancing the reacted liquid sample to the second chamber of the biosensor; and measuring a detectable signal in the second chamber of the biosensor. The detectable signal can indicate the presence and/or amount of the target analyte in the liquid sample. The method can include filling the first chamber of the biosensor with the liquid sample. The liquid sample can remain in the first chamber of the biosensor for a period of time (for example, for a pre-determined period of time) before it can advance to the second chamber. In some embodiments, deriving the result can include producing at least one result selected from the group consisting of, for example, a qualitative result as to whether the target analyte is present in the sample, a semi-quantitative result which gives an approximate range of the concentration or the target analyte in the sample, a quantitative estimate of the concentration of the target analyte in the liquid sample, and the like. The form of the target analyte can include, for example, an active form, an inactive form, a defective form, or the like.

Some embodiments of the invention include a method of fabricating a biosensor disclosed herein.

### BRIEF DESCRIPTION OF THE DRAWINGS

FIG. 1 is a schematic cross-sectional view of a strip disclosed herein in which a sample is added to a reaction chamber.

FIG. 2 is the strip of FIG. 1 where the sample has reacted and moved to a detection chamber.

### DETAILED DESCRIPTION

In some embodiments, the numbers expressing quantities of ingredients, properties, such as molecular weights, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Embodiments of the invention are directed towards a device (biosensor) and method for detecting a target analyte and/or its activity (e.g., the activity of one or more enzymes) in a liquid sample where the mode of action of the target analyte is to cleave a bond. In some embodiments, the action of an enzyme of interest (target analyte) is selectively detected via cleavage of a linker that is specifically cleaved by the target analyte. The device and method can be simple to apply at point of care.

Some embodiments of the inventions disclosed herein include a novel method and device (biosensor) for detecting an enzyme with a cleavage mode of action (target analyte) that is suitable for a point-of-care or laboratory test device or system. The device can include a single-use test element, herein referred to as a strip (biosensor), and a reusable (e.g., electronic, optical, or the like) instrument portion, herein referred to as a meter. The strip (biosensor) can provide the chemistry to generate a liberated probe species if the target analyte, or a form thereof (e.g., an active form), is present; and the meter can provide a stimulation to generate a detectable signal from the detectable species (e.g., the liberated probe species or a detectable reaction product), and/or measure the signal, and/or analyze the signal and report or convey the test result, either locally or remotely through communication to other devices.

The strip (biosensor) can include at least two chambers, a first chamber and a second chamber. In use, a liquid sample can be caused to fill the first chamber. In the first chamber, the presence of target analyte can cause the liberation into the liquid sample of a label species (probe species) that can be either directly detectable or can further react to lead to a reaction product that is detectable in the second chamber. The liberation process can be allowed to proceed (e.g., for a pre-determined period of time). Subsequently, the reacted liquid sample containing the liberated probe species can be transferred to the second chamber where the detection signal can be read, either directly from the label species (liberated probe species), or from a reaction product generated by a reaction (detection reaction) that the label species (probe species) undergoes in the second chamber. By quantifying the amount of label (e.g., the liberated probe species or the reaction product) present in the second chamber the activity of the target analyte in the sample can be quantified or semi-quantified.

In some embodiments of the invention described herein, the device is adapted such that the presence and/or amount of free or liberated probe species in the liquid sample is dependent upon cleavage by the target analyte. This can represent a significant departure from the prior art. Merely by way of example, it represents a novel and/or advantageous way of detecting a species of interest without having to rely on a binding reaction. It can achieve low background signals as the immobilisation or retardation of the probe species by, e.g., a covalent bond, can be very strong before the probe species is liberated by a cleavage reaction by the target analyte. Accordingly, there is a low likelihood of having free or liberated probe species in the first chamber (e.g., reaction chamber) and/or in the second chamber (e.g., the detection chamber) in the absence of the target analyte.

In contrast, if a binding reaction, e.g., a competitive binding assay, a displacement binding assay, or the like, is involved, a binding species with relatively low affinity to a binding partner can be used. Merely by way of example, in a displacement assay, a reporter complex including a binding species and a probe (e.g., a detectable probe) is bound to a binding partner prior to introduction of a liquid sample, the binding affinity of the binding species is lower than that of a target analyte to the binding partner. The binding species (and the target analyte) and the binding partner can include an antigen and an antibody, respectively, or vice versa. The reporter complex can be displaced by the target analyte in the liquid sample, and the free reporter complex can be measured. Due to the relatively low affinity of the reporter complex to the binding partner, the reporter complex can be disassociated from the binding partner in the absence of the target analyte, thereby generating a relatively high background signal, and/or a relatively high measurement error.

Additionally, in a device involving a binding reaction in small volumes of liquid, such as typical point-of-care blood tests, a species (e.g., a reporter complex) including a probe (e.g., a detectable probe) may need to diffuse some distance, thus limiting either the size that the species (e.g., a reporter complex) including a probe (e.g., a detectable probe) can be or the rapidity of the test. In some embodiments of the instant invention, the probe species is linked to a linker via a bond prior to introduction of a liquid sample. If the liquid sample includes the target analyte, the linker can be cleaved by the target analyte with specificity. There is no need for the probe species to diffuse or otherwise move within the liquid sample for the cleavage to occur. Thus, the probe species can be very large without affecting the functionality of the device. For example, a copy of the probe species (linked to a linker) can include a polymer of enzymes (i.e. multiple copies of an enzyme conjugated or otherwise joined together), or multiple copies of an optically or electrically active molecule conjugated or otherwise jointed together. In such embodiments, liberation of one copy of the probe species can lead to multiple copies of a detectable species (e.g., multiple copies of an optically or electrically active molecule that can be detectable directly; or multiple copies of an enzyme that can participate in multiple detection reactions, thereby generating multiple copies of a detectable reaction product). It can often be advantageous to have such a large probe species (e.g., a copy of a probe species including multiple copies of an enzyme that can catalyse at least one detection reaction, or multiple copies of an optically and/or electrically active molecules) as it can have increased activity in the detection chamber and thus increase the sensitivity and/or accuracy of the device. Moreover, the amount of the liquid sample to run the measurement using such a device disclosed herein can be reduced because the amount of the target analyte needed to generate a detectable signal can be reduced.

The terms “device,” “strip,” and “biosensor” are used interchangeably herein unless otherwise stated. The probe species are also referred to as the label species. The probe species or the labeled species can have at least two statuses in the biosensor, retained in the first chamber via a linker, or liberated. A detectable species can be the liberated probe species, or can be a detectable reaction product generated in a detection reaction the liberated probe species undergoes in a second chamber of the biosensor.

A target analyte can have an active form, and an inactive form. The target analyte in its inactive form does not have its normal cleavage function, and therefore cannot cleave the linker to liberate the probe species. As used herein, the term target analyte indicates it is in its active form unless otherwise stated.

Some embodiments of the inventions include a biosensor for detecting a target analyte in a liquid sample. The biosensor can include at least a first chamber and a second chamber, wherein the first chamber and the second chamber can be in fluid communication, wherein the first chamber can include a probe species, wherein the probe species is retained in the first chamber via a linker, wherein the target analyte can be capable of cleaving the linker to liberate the probe species into the liquid sample in the first chamber, wherein the biosensor is configured to move the liquid sample (including the liberated probe species if applicable) from the first chamber to the second chamber, and wherein the liberated probe species (if present in the liquid sample) can be detected in the second chamber via a detection mechanism.

In some embodiments, the biosensor can include a first chamber and a second chamber. In some embodiments, the first chamber can include a reaction chamber, and the second chamber can include a detection chamber. The first chamber and the second chamber can be in fluid communication.

In some embodiments, the first chamber can include the probe species. The probe species can be retained in the first chamber via the linker. If the target analyte is present in the liquid sample, it can cleave the linker to liberate the probe species, so that the probe species is free to move with the liquid sample. After a period of time (e.g., a pre-determined period of time) in the first chamber to allow the cleavage to occur, the reacted liquid sample can be transferred to the second chamber, transporting the liberated probe species with it, but leaving the probe species linked to an intact linker (i.e. not cleaved) in the first chamber. The cleavage reaction can be specific. If there is no target analyte in the liquid sample (e.g., its concentration or amount is below the detectable level) or the target analyte is not active or functioning, the cleavage reaction may not occur, and the probe species can be retained in the first chamber.

In some embodiments, the probe species can be retained (e.g., immobilized or retarded) in the first chamber (e.g., reaction chamber) of the biosensor (strip) via the linker by any suitable method. Merely by way of example, the linker can be directly absorbed to, tethered to, or supported on one or more internal surfaces of the first chamber; or it can be tethered to or supported on the surface of a separate support, where the support can be prevented or retarded from entering the second chamber (e.g., detection chamber). The linker can be attached to (absorbed to, tethering to, supporting on, or the like) a surface (e.g., one or more internal surface of the first chamber, the surface of a separate support, or the like) by any method that can yield a sufficiently stable bond such that at equilibrium there is only a small amount of dissociated probe species in the liquid sample in the absence of the target analyte. As used herein, a small amount of the dissociated probe species indicates that the amount is below the level of the lower detection limit of the device or system. Suitable methods can include, for example, covalent bonding to one or more groups located on the surface (e.g., one or more internal surface of the first chamber, the surface of a separate support, or the like), high affinity non-covalent bonding to one or more groups located on such a surface, or the like. The high affinity non-covalent bond can include, for example, a streptavidin/biotin bond, a thiol/gold bond, or the like.

In some embodiments, the separate support can include a bead. The bead can be magnetic, and the probe species plus the linker construct attached to the bead can be retained in the first chamber by a magnetic force. The biosensor can include, for example, a magnet, or the like that can generate a magnetic force. Merely by way of example, the construct can be tethered to or supported on a separate support as disclosed in U.S. Patent Application Publication No. US 20060134713 entitled BIOSENSOR APPARATUS AND METHODS OF USE, which is hereby incorporated by reference. For example, in some embodiments, the construct can be tethered to polymer coated magnetic core beads such as PROMAG or BIOMAG beads from BANGS LABORATORIES, INC., or SPHEROTECH beads from SPHEROTECH, INC. The benefits of attaching (tethering, supporting, or the like) the construct to a separate support can include, for example, easier fabrication. For example, as the construct can be attached to the support independently of the main strip fabrication processes, which can allow a broader choice of conditions and schemes for performing the attachment and ease of washing to remove unattached constructs and/or its constituents (e.g., the linker and/or the probe species). Additional benefits can include that the support can also provide a greater surface area for attachment, increasing the achievable loading of construct in the first chamber (e.g., reaction chamber).

In some embodiments, the probe species can be linked to the linker via a bond. The bond can include at least one bond selected from the group consisting of, for example, a covalent bond, a non-covalent bond, and the like. Exemplary non-covalent bonds can include, for example, a bond due to a Van der Waals interaction, such as, for example, a hydrogen bond, an electrostatic bond, or the like.

In some embodiments, the biosensor can be suitable for detecting a target analyte in a liquid sample. The liquid sample can be, for example, whole blood, plasma, serum, mucus, urine, tissue prep in liquid form. There can be one or more preparation steps undertaken on the sample before the sample is ready for use with the device. The steps taken can depend on type and availability of the target analyte in the sample. For example, if the target analyte is contained within cells in the sample, at least one of the preparation steps can include making the target analyte available by way of, such as, for example, lysing the cells.

In embodiments disclosed herein, the target analyte can cleave the linker to liberate the probe species. The cleavage can be specific to the target analyte. To liberate the probe species in the first chamber (e.g., reaction chamber) specifically in the presence of the target analyte, the linker that anchors the probe species to a surface (e.g., one or more internal surfaces of the first chamber, a surface of a separate support, or the like) in the first chamber (e.g., reaction chamber) can be chosen such that it can be cleaved specifically by the target analyte and not at a significant rate by other species that can be expected to be present in test liquid samples. The linker can include a natural substrate for the target analyte that can be cleaved. The linker can include a synthetic version or analogue of natural substrate of the target analyte.

In some embodiments, the probe species can include a species that can be detected in the second chamber (e.g., detection chamber). The detection can use, for example, an optical method, an electrochemical method, or the like. In some embodiments, the probe species can include at least one species selected from the group consisting of, for example, an optically molecule, an enzyme, an electrically active molecule, and the like. For example, the optically active molecule can include one molecule that can absorb light, emit light when excited, such as a fluorescent molecule, a phosphorescent molecule, a chemiluminescent molecule, or the like. More exemplary probe species can be found in, for example, PCT Patent Application Publication Nos. WO 2002/008763 entitled Immunosensor, and WO 2010/004436 entitled ENHANCED IMMUNOASSAY SENSOR; and U.S. Patent Application Publication Nos. US 20030180814 entitled DIRECT IMMUNOSENSOR ASSAY and US 20060134713 entitled BIOSENSOR APPARATUS AND METHODS OF USE, each of which is hereby incorporated by reference.

In some embodiments, the biosensor can be suitable for detecting a target analyte by way of the action of the target analyte cleaving a chemical bond of the linker (referred to as cleaving or cleavage for the purposes of simplicity), thereby liberating the probe species. The target analyte can include an enzyme. The cleavage to liberate the probe species can be specific to the target analyte. Examples of a suitable enzyme can include a chymotrypsin, a pepsin, a papain, an isopeptidase, a thrombin, a lactase, a maltase, a sucrase, an amylase, a pappalysin-2, a lysozyme, a protease, and a matrix metalloproteinase, or the like.

The liberated probe species can move to the second chamber with the (reacted) liquid sample and can be detected in the second chamber (e.g., the detection chamber). In some embodiments, the probe species can be detected directly in the second chamber via the detection mechanism. Merely by way of example, the probe species can include an optically active molecule, an electrically active molecule, or the like. The presence and/or amount of such liberated probe species can be detected directly in the second chamber.

In some embodiments, the liberated probe can be detected indirectly in the second chamber (e.g., the detection chamber). For example, the liberated probe can undergo a reaction (e.g., the detection reaction) with a reagent in the second chamber (e.g., the detection chamber) to produce a reaction product that can be detected via the detection mechanism. In some embodiments, the detection reaction can include at least one intermediate reaction, and/or generate at least one intermediate reaction product. In some embodiments, the second chamber can include one or more reagents. The reagent(s) can participate in the detection reaction or at least one of the intermediate reaction(s) in the presence of the liberated probe species to generate the detectable reaction product. The reagent can include at least one reagent selected from the group consisting of, for example, a substrate, a mediator, a cofactor, a buffer, an electrochemical species, and the like. The cofactor can include, for example, fyrroloquinoline quinone, flavin adenine dinucleotide, flavin mononucleotide, nicotinamide adenine dinucleotide, or the like. The buffer can include, for example, phosphate, mellitate, or the like. The mediator can include, for example, dichlorophenolindophenol, a complex between a transition metal and a nitrogen-containing heteroatomic species, ferricyanide, or the like. An electrochemical species can include, for example, Ag/AgCl redox pair, Zn/ZnCl₂, or the like. The second chamber can include more than one reagent. The substrate can include an enzyme substrate. To improve the sensitivity and/or accuracy of the device, it can be desired that a copy of the target analyte can cause production and detection of more than one copy of the detectable reaction product or signal. In some embodiments, one linker can link to a probe species including multiple copies of an enzyme (that can catalyze the detection reaction or one or more intermediate reactions) or an optically or electrically active molecule. In some embodiments, one copy of the probe species is able to produce more than one, and most preferably a multiplicity of copies of species that can be detected. For example the probe species can include an enzyme or otherwise have an enzymatic action, where it can react with other one or more reagents in the detection chamber to produce one or more than one copy of a reaction product that is detectable by the device or system. The type of enzyme that is suitable can depend upon the detection mechanism. For example, for an optical detection method, a reaction product of the enzymatic action of the liberated probe species can have a detectable optical property. If, for example, the absorbance of light is the detection signal, then a reaction product that can absorb light at an appropriate wavelength can be produced. In such embodiments, an enzyme such as, for example, glucose oxidase or the like, can be employed to react with glucose present in the detection chamber to produce, among other things, hydrogen peroxide, which can further react with horseradish peroxidase and a dye to produce a coloured species. If chemiluminescence microscopy is used, then an enzyme such as, for example, luciferase, or the like, can be used to produce a change to the chemiluminescence of the liquid sample. If a potentiometric method, a coulometric method, an amperometric method, or the like, is used, then an enzyme such as, for example, glucose oxidase, glucose dehydrogenase, or the like, can be used, where the enzyme reacts with a substrate and mediator in the detection chamber to produce a redox species that can be oxidized or reduced at an electrode. In some embodiments of the invention, the probe species can itself be an enzyme that can react with a substrate in the second chamber to form a detectable species. One probe species liberated by the target analyte in the first chamber can result in many copies of a detectable species being generated in the second chamber, thus increasing the sensitivity and/or speed of the detection assay. This can be because that the liberated probe species as an enzyme is not consumed in the reaction and can be recycled to catalyze more of the reaction in the second chamber.

In some embodiments, the detection mechanism can include at least one mechanism selected from the group consisting of, for example, reflectance spectroscopy, transmission spectroscopy, fluorometry, turbidimetry, chemiluminescence microscopy, coulometry, amperometry, potentiometry, and the like. In some embodiments, amperometry can be advantageous due to the relative simplicity of implementation in a small electronic device and the suitability to detection in a whole blood sample. Exemplary detection mechanism can be found in, for example, PCT Patent Application Publication Nos. WO 2002/008763 entitled Immunosensor, and WO 2010/004436 entitled ENHANCED IMMUNOASSAY SENSOR; and U.S. Patent Application Publication Nos. US 20030180814 entitled DIRECT IMMUNOSENSOR ASSAY and US 20060134713 entitled BIOSENSOR APPARATUS AND METHODS OF USE, each of which is hereby incorporated by reference. Merely by way of example, in U.S. Patent Application Publication No. US 20060134713 entitled BIOSENSOR APPARATUS AND METHODS OF USE, a two-chamber strip is described that is directed towards the selective detection of species using the binding of species such as antibodies or antigens. In the first chamber the binding reactions occur that are dependent on the presence of the analyte of interest, whereupon the fluid is transferred to a second chamber where a probe species can be detected.

The second chamber can be distal to the first chamber. The first chamber can include one or more walls to form the chamber including one or more internal surfaces. The second chamber can include one or more walls to form the chamber including one or more internal surfaces. The second chamber is configured to be suitable for the desired detection mechanism. In some embodiments in which the desired detection mechanism is optical detection, one or more walls of the second chamber can be transparent to the optical stimulus and the generated optical signal to achieve the detection.

In some embodiments in which the desired detection mechanism is electrochemical detection, the second chamber can include at least two electrodes. One or more internal surfaces of the second chamber can be coated with an electrically conductive material. On at least one internal surface of the second chamber, the electrically conductive material can be co-extensive with the internal surface of the second chamber on which the electrically conductive material is coated. On at least one internal surface of the second chamber, the electrically conductive material can cover an area smaller than that of the internal surface of the second chamber on which the electrically conductive material is coated. The two or more electrodes can be located on the same internal surface of the second chamber. The two or more electrodes can be located on the different internal surfaces of the second chamber. The two or more electrodes can be electrically insulating to each other. The second chamber can include a break in the electrically conductive layer that can serve to define at least one edge of the electrode in the second chamber. At least one electrode can include carbon, gold, palladium, platinum, iridium, or the like, or an alloy thereof, such as, for example, tin oxide, indium oxide and mixed indium oxide/tin oxide, or the like.

The biosensor can include more than two chambers. Merely by way of example, the biosensor can include a filling chamber or passage. The filling chamber or passage can be in fluid communication with the first chamber to transfer a liquid sample from a filling port to the first chamber. The filling chamber or passage can be proximal to the first chamber, while the second chamber can be distal to the first chamber. The filling chamber or passage can include one or more walls to form the chamber including one or more internal surfaces.

The first chamber can include a filling port at the proximal end. The first chamber can include a mechanism to measure and/or signal filling with a liquid sample. In some exemplary embodiments, one or more walls of the first chamber can be transparent to visible light. The advance of the liquid sample within the first chamber to a desirable extent can be visible to a user. In some exemplary embodiments, the advance of the liquid sample within the first chamber of the biosensor can be determined using an optical detection. For example, the change in an optical parameter (e.g., light absorption or deflection) before and after the liquid sample reaches a desirable position in the first chamber can trigger a signal (e.g., an audible and/or visual signal to alert the user) or a control signal. In some exemplary embodiments, the first chamber can include a circuit, wherein the filling of the first chamber with a liquid sample to a desirable extent can generate an electrical signal. The electrical signal can be converted to a signal (e.g., an audible and/or visual signal) to alert a user or a control signal. The filling chamber and/or the second chamber can include a mechanism to measure and/or signal filling with a liquid sample.

The biosensor can be configured to move the liquid sample via capillary action. The biosensor can be configured to move the liquid sample from the first chamber to the second chamber via capillary action. The capillary force that the first chamber and/or the second chamber can generate for driving the movement of the liquid sample within the biosensor can be affect by, for example, the dimension of the first chamber compared to that of the second chamber, the surfactant on one or more internal surfaces of the first chamber compared to that on one or more internal surfaces of the second chamber. Merely by way of example, the first chamber has a first height, and the second chamber has a second height that is smaller than the first height, thereby generating a larger capillary force to attract the liquid sample into the second chamber compared with that generated by the first chamber. The filling of the first chamber by the liquid sample can compress the air trapped within the biosensor, thereby generating a back pressure to prevent further advance of the liquid sample into the detection chamber until activation by, for example, opening a vent in the second chamber to release the trapped air and therefore the back pressure. The vent can be located at the distal end of the second chamber. Depending on the configuration of the first chamber and the second chamber, the activation can be achieved by application of an external force (e.g., a positive pressure, a centrifuge force) to the liquid sample, breaking the surface tension of the liquid sample. Other structural features can be employed in the biosensor to achieve the filling of the biosensor in a controlled manner. Disclosure of such features can be found in. for example, PCT Patent Application Publication Nos. WO 2002/008763 entitled Immunosensor, WO 2007/096730 entitled FLUID TRANSFER MECHANISM, and WO 2010/004436 entitled ENHANCED IMMUNOASSAY SENSOR; U.S. Patent Application Publication Nos. US 20030180814 entitled DIRECT IMMUNOSENSOR ASSAY and US 20060134713 entitled BIOSENSOR APPARATUS AND METHODS OF USE; and U.S. Pat. No. 4,426,451 entitled MULTI-ZONED REACTION VESSEL HAVING PRESSURE-ACTUATABLE CONTROL MEANS BETWEEN ZONES, and U.S. Pat. No. 4,863,498 entitled CAPILLARY FLOW DEVICE, each of which is hereby incorporated by reference.

In some embodiments, the biosensor disclosed herein can be used for at least one measurement to determine presence of the target analyte in the liquid sample, quantity of the target analyte in the liquid sample, presence of a form of the target analyte in the liquid sample, and quantity of the target analyte in the liquid sample. The form of the target analyte comprises an active form, an inactive form, or a defective form.

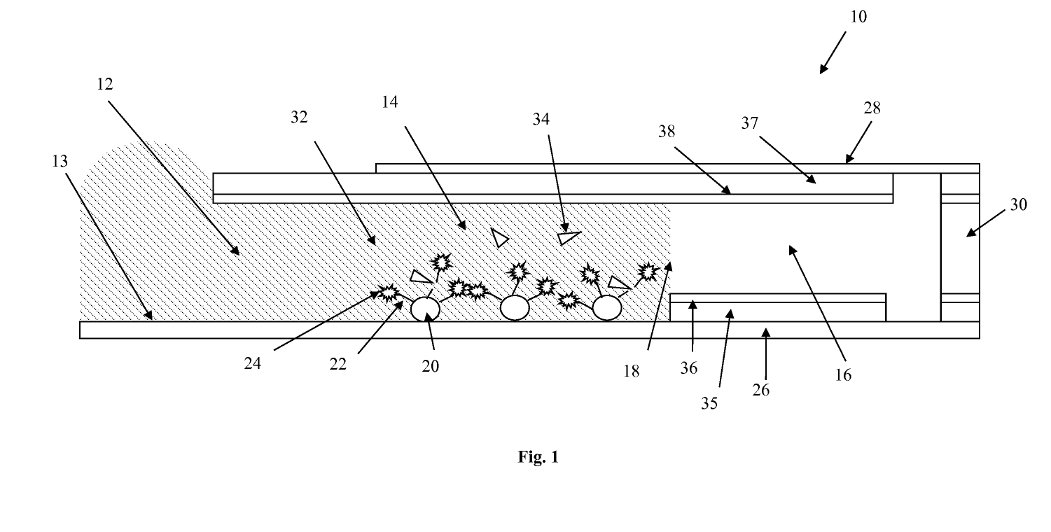

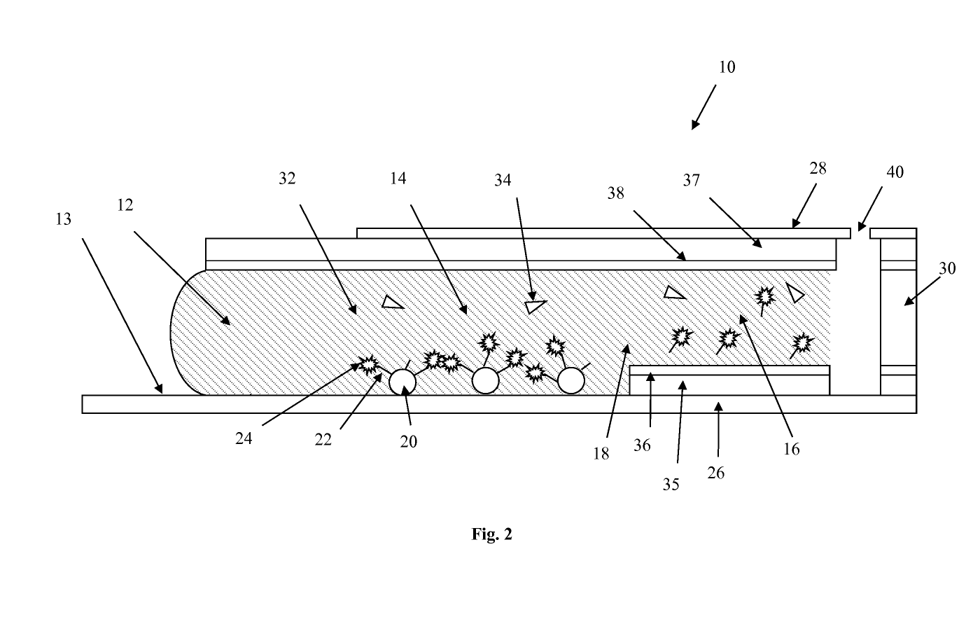

Some exemplary embodiments of the biosensor described herein are illustrated in FIGS. 1 and 2. Strip 10 can include inlet 12, reaction chamber 14, detection chamber 16 and fluid connection 18 between reaction chamber 14 and detection chamber 16. Inlet 12 can include ledge 13 on to which inlet 12 opens. Located within reaction chamber 14 can include support 20 to which linker 22 and probe species 24 are attached. In the exemplary embodiments shown in FIGS. 1 and 2, support 20 can include a magnetic bead and be located in reaction chamber 14; linker 22 can be attached to (e.g., tethered to, supported on, or the like) support 20 located in reaction chamber 14. The strip can contain other chambers (not shown). For example, a filling chamber or passage can be included to transfer liquid sample 32 from a filling port (inlet 12) to reaction chamber 14.

Reaction chamber 14 can be formed between first sealing sheet 26 and second sealing sheet 28, and support layers 35 and 37, which are spaced apart by one or more spacers, e.g., middle sheet 30. Detection chamber 16 can be formed between first sealing sheet 26 and second sealing sheet 28, and support layers 35 and 37, which can be spaced apart by one or more spacers, in the exemplary embodiments, middle sheet 30. It is understood that more than one middle sheet 30 can be included depending on the desired configuration. One or more electrically conductive materials can be supported on support layers 35 and 37, respectively, to form electrodes 36 and 38. The electrically conductive materials can be co-extensive to support layers 35 and 37, respectively. At least one of the electrically conductive materials can cover an area smaller than that of support layers 35 and 37, respectively. One or more of spacer 30, support layers 35 and 37, first sealing layer 26, and second sealing layer 28 can be electrically insulating.

FIG. 1 illustrates that liquid sample 32 is filling inlet 12 of reaction chamber 14. When ready a drop of liquid sample 32 can be placed onto, for example, ledge 13 on to which inlet 12 opens, and the liquid sample can be transported from the filling port (inlet 12) to reaction chamber 14 by capillary action. Optionally additional sample 32 can be located at an entrance (e.g., on ledge 13) to inlet 12 to act as a sample reservoir. Sample 32 includes target analyte 34, which can be detected. Linker 22 is attached on substrate 20 and linked to probe species 24. In the exemplary embodiments shown in FIGS. 1 and 2, probe species 24 is retained in reaction chamber 14 via linker 22, where the target analyte can cleave linker 22 and can thus liberate probe species 24. In FIG. 1 target analyte 34 is shown cleaving linker 22 to liberate probe species 24 into the liquid sample 32. In FIG. 2, reacted liquid sample 32 has moved from reaction chamber 14 to detection chamber 16 upon opening of vent 40 at the distal end of detection chamber 16. Liberated probe species 24 can move with sample 32 into detection chamber 16 as described below, placing liberated probe species 24 in detection chamber 16. Probe species 24 that are not liberated by cleavage and therefore remain linked to linker 22 can be retained in reaction chamber 14. Electrodes 36 and 38, located on support layers 35 and 37, respectively, form the walls of detection chamber 16, and in use can be in electrical communication with a power source to provide or measure a potential difference across detection chamber 16. In some embodiments, in use the electrodes can be connected to a meter having a power source and a computer to determine timing and amount of potential difference to be applied.

Some embodiments of the invention include a system for detecting a target analyte in a liquid sample. The system can include a biosensor described herein. The system can also include a meter. The meter can generate a stimulation to facilitate direct detection of the liberated probe species, or indirect detection thereof (though detection of a reaction product generated by a detection reaction the liberated probe undergoes in the second chamber).

In some embodiments, the system can generate a stimulation for the detection reaction. The stimulation can include at least one stimulation selected from the group consisting of, for example, an electrical stimulation, an optical stimulation, and the like. The electrical stimulation can include at least one stimulation selected from the group consisting of, for example, a current, a potential, and the like. The optical stimulation can include a light including one or more wavelengths. In some embodiments, the stimulation can be constant with time. In some embodiments, the stimulation can vary with time. In some embodiments, the stimulation can be generated by the meter.

In some embodiments, it can be advantageous to maintain the first chamber (e.g., reaction chamber) and/or the second chamber (e.g., detection chamber) of the strip (biosensor) at a controlled temperature. The rate of the target analyte cleavage reaction with the linker can be temperature-dependent. To facilitate the quantification of the amount and/or activity of the target analyte it can be desirable to be able to correlate the cleavage reaction rate to the activity of the target analyte using a known relationship. Controlling the temperature in the first chamber (e.g., reaction chamber) can remove temperature as a variable in inferring with a target analyte activity from a cleavage rate. In some embodiments, in the second chamber (e.g., detection chamber) the signal measured can be dependent upon the temperature, for example, when the detection is based on the rate of a detection reaction that a probe species including an enzyme is involved. The temperature of the first chamber (e.g., reaction chamber) and/or the second chamber (e.g., detection chamber) can be controlled by any suitable method. One suitable method is to place the strip or biosensor in the meter so that it is in contact with a heater or heating element, where the heating is controlled to maintain a desired temperature. The desired temperature can be a constant temperature, or a variable temperature.

In some embodiments, the system can include a temperature control apparatus. The temperature control apparatus can include, for example, a heater, a heating element, a cooling element, or the like. The temperature can be maintained at a temperature suitable for the cleavage reaction to occur, and/or that suitable for the detection reaction to occur. For example, the temperature can be below 100° C., or below 80° C., or below 60° C., or below 50° C., or below 45° C., or below 42° C., or below 40° C., or below 38° C., or below 37° C., or below 35° C., or below 30° C., or below 25° C. In some embodiments, the system can include a temperature measurement apparatus. The temperature measurement apparatus can include, for example, a thermometer, or the like. The system can include a temperature signalling apparatus to signal the temperature within the system. In some embodiments, the temperature signalling apparatus can generate a signal when the temperature within the system is suitable for detecting the target analyte. In some embodiments, the temperature signalling apparatus can generate a signal when the temperature within the system is not suitable for detecting the target analyte. The signal can include an audible signal or a visual signal. The system can include one or more of the temperature control apparatus, the temperature measurement apparatus, the temperature signalling apparatus described herein, or the like. One or more of the temperature control apparatus, the temperature measurement apparatus, the temperature signalling apparatus described herein, or the like, can be located within the meter.

In some embodiments, the system can include a timing mechanism. The timing mechanism can include a mechanism to record the starting of the reaction in the first chamber of the biosensor. In some exemplary embodiments, the advance of the liquid sample within the first chamber of the biosensor to a desirable extent can be visible to a user. In some exemplary embodiments, the advance of the liquid sample within the first chamber of the biosensor can be determined using an optical detection. For example, the change in an optical parameter (e.g., light absorption or deflection) before and after the liquid sample reaches a desirable position in the first chamber can trigger a signal (e.g., an audible and/or visual signal to alert the user); or such a change in an optical parameter can trigger a control signal to the system (e.g., a control signal to the meter). In some exemplary embodiments, the first chamber can include a circuit, wherein the filling of the first chamber with a liquid sample to a desirable extent can generate an electrical signal. The electrical signal can be converted to a signal (e.g., an audible and/or visual signal) to alert the user, or a control signal to the system (e.g., a control signal to the meter). In some embodiments, upon receipt of a signal, a user can manually record the time when the liquid sample fills the first chamber to a desirable extent and the cleavage reaction in the first chamber starts. In some embodiments, a control signal can trigger an automated recordation of the time when the liquid sample fills the first chamber to a desirable extent and the cleavage reaction in the first chamber starts.

In some embodiments, the timing mechanism can include a mechanism to control the time during which the cleavage reaction occurs in the first chamber and when the reacted liquid sample can advance to the second chamber. The mechanism can include, for example, a timer. After the cleavage reaction in the first chamber proceeds for a pre-determined time, the timer can generate a signal (e.g., an audible and/or visual signal) to alert the user, or a control signal to the system (e.g., a control signal to the meter). In some embodiments, upon receipt of a signal, a user can manually activate the advance of the reacted liquid sample to the second chamber. In some embodiments, a control signal can trigger an automated activation of the advance of the reacted liquid sample to the second chamber. In some embodiments, the activation can include at least one mechanism selected from the group consisting of, for example, opening a vent at the distal end of the second chamber, application of an external force (e.g., a positive pressure, a centrifuge force) to the liquid sample, breaking the surface tension of the liquid sample, and the like. Exemplary method of temporarily stopping and resuming the flow of the liquid sample within the biosensor can be found in, for example, PCT Patent Application Publication Nos. WO 2002/008763 entitled Immunosensor, WO 2007/096730 entitled FLUID TRANSFER MECHANISM, and WO 2010/004436 entitled ENHANCED IMMUNOASSAY SENSOR; U.S. Patent Application Publication Nos. US 20030180814 entitled DIRECT IMMUNOSENSOR ASSAY and US 20060134713 entitled BIOSENSOR APPARATUS AND METHODS OF USE; and U.S. Pat. No. 4,426,451 entitled MULTI-ZONED REACTION VESSEL HAVING PRESSURE-ACTUATABLE CONTROL MEANS BETWEEN ZONES, and U.S. Pat. No. 4,863,498 entitled CAPILLARY FLOW DEVICE, each of which is hereby incorporated by reference. The system can include one or more of the timing mechanisms described herein, or the like. One or more of the timing mechanisms described herein, or the like, can be located within the meter.

In some embodiments, the system can include a mechanism to process the detected signal to derive a result in a desired format. For example, the desired format can include, for example, a qualitative result as to whether the target analyte is present in the sample, a semi-quantitative result which can give an approximate range of the concentration or the target analyte in the sample, a quantitative estimate of the concentration of the target analyte in the sample, or the like. The system can include one or more mechanisms including, for example, a speaker, a screen, or a printer, or the like, to report or convey the result in a desired format. The mechanism to process the detected signal to derive a result in a desired format and/or the mechanism to report or convey the result in the desired format can be located within the meter.

Merely by way of example, a system for detecting a target analyte in a liquid sample includes two ore more electrodes, wherein each of at least two of the two or more electrodes is electrically connected to a contact pad, wherein the contact pads can be electrically connected to the meter. Exemplary configurations of the electrodes and/or contact pads can be found in, for example, PCT Patent Application Publication Nos. WO 2002/008763 entitled Immunosensor, WO 2007/096730 entitled FLUID TRANSFER MECHANISM, and WO 2010/004436 entitled ENHANCED IMMUNOASSAY SENSOR; and U.S. Patent Application Publication Nos. US 20030180814 entitled DIRECT IMMUNOSENSOR ASSAY and US 20060134713 entitled BIOSENSOR APPARATUS AND METHODS OF USE, and US 20060266644 entitled METHOD AND APPARATUS FOR ELECTROCHEMICAL ANALYSIS; and U.S. Pat. No. 8,192,599, entitled METHOD AND APPARATUS FOR ELECTROCHEMICAL ANALYSIS, each of which is hereby incorporated by reference. The system can include one or more of the temperature control apparatus, the temperature measurement apparatus, the temperature signalling apparatus described herein, or the like. The system can include one or more of the timing mechanisms described herein. The system can include a similar timing mechanism for the detection in the second chamber. The system can include one or more methods or apparatuses including, for example, a speaker, a screen, or a printer, or the like, to report or convey the result in a desired format.

Some embodiments of the invention include a system that can include a strip, for example, strip 10 as shown in FIGS. 1 and 2, wherein the strip is intended to be used once, and a meter (not shown) that is intended to be used many times to perform multiple tests. The strip can be introduced into the meter and sample introduced into the strip. The strip can contain some or all of the reagents needed to perform the required chemistry. The meter can be responsible for applying any external simulation to the strip for measuring the output signal from the strip, deriving a result from the signal and presenting the result.

In some embodiments, the strip can include at least two chambers. In the first chamber, in the presence of the target analyte or analytes in the liquid sample, one or more reactions can occur that can result in a probe species being liberated so as to become mobile in the liquid sample. This reaction can be allowed to proceed for a period of time (e.g., a pre-determined period of time). This chamber is termed the reaction chamber. Subsequently, the reacted liquid sample from the reaction chamber containing any liberated probe species can be transferred to the second chamber, termed the detection chamber. In this chamber the liberated probe species can generate a signal that can be detectable or readable by the meter, or can react with one or more other reagents to generate one or more reaction products that can be detectable or readable by the meter. By transferring the reacted liquid sample from the reaction chamber to the detection chamber, the liberated probe species can be carried to the detection chamber. If the liberated probe species is indirectly detected through the detection of one or more further reaction products, then the reagents to react with the liberated probe species and produce the one or more reaction products can be dried into the detection chamber during strip manufacture. In some embodiments, the reagent(s) can be applied to the chamber in a liquid form, and then dried. In some embodiments, the reagent(s) may not be dried but can be of a form that can remain within the reaction chamber during the reaction, for example, a gel.

Some embodiments of the invention include a method of using a system described herein for detecting a target analyte in a liquid sample. The system can include a biosensor described herein. The biosensor can include a first chamber and a second chamber. The first chamber can include a probe species, wherein the probe species can be retained in the first chamber via a linker, wherein the target analyte can cleave the linker to liberate the probe species into the liquid sample in the first chamber, wherein the liberated probe species in the liquid sample can be transferred to the second chamber, and wherein the liberated probe species can be detected in the second chamber via a detection mechanism. The system can also include a meter. The meter can generate a stimulus to facilitate direct detection of the liberated probe species, or indirect detection thereof (through detection of a reaction product generated by a detection reaction the liberated probe undergoes in the second chamber). The method can produce a qualitative result as to whether the target analyte is present in the sample, a semi-quantitative result which can give an approximate range of the concentration or the target analyte in the sample, a quantitative estimate of the concentration of the target analyte in the liquid sample, or the like. The method can include providing the liquid sample; allowing the liquid sample to remain in the first chamber of the biosensor to generate a reacted liquid sample; advancing the reacted liquid sample to the second chamber of the biosensor; and measuring a detectable signal in the second chamber of the biosensor. If the liquid sample includes the target analyte, the target analyte can cleave the linker to liberate the probe species into the liquid sample in the first chamber. The advancing of the reacted liquid sample to the second chamber can move the liberated probe species therein to the second chamber for direct or indirect detection. The cleavage reaction can proceed for a period of time, e.g., a pre-determined period of time. The pre-determined time can depend from the cleavage reaction employed in the system. The pre-determined time can be shorter than 10 minutes, or shorter than 8 minutes, or shorter than 6 minutes, or shorter than 4 minutes, or shorter than 2 minutes, or shorter than 1 minute, or shorter than 40 seconds, or shorter than 30 seconds, or shorter than 20 seconds. The method can further include filling the first chamber of the biosensor with the liquid sample. The method can further including deriving a result in a desired format from the detectable signal.

Merely by way of example, a method for using the system is described with reference to the exemplary embodiments shown in FIGS. 1 and 2. Strip 10 containing the dry reagents can be placed in a meter (not shown) and the strip allowed to warm to the desired temperature. When ready a drop of liquid sample 32 can be placed onto, for example, ledge 13 on to which inlet 12 opens, whereupon the liquid sample can be transported from the filling port (inlet 12) to reaction chamber 14 by capillary action. Upon the filing of reaction chamber 14 the meter (not shown) can automatically recognise that sample has been applied and can start a timer. For example, the user can push a button on the meter to indicate that a sample has been added. The sample can be prevented from substantially entering detection chamber 16 due to air trapped in detection chamber 16. Once in reaction chamber 14, the sample dissolves or mobilises the reagents located in reaction chamber 14. If active target analyte 34 is present it can cleave linker 22 anchoring probe species 24 to a surface such as that of support 20. This reaction can be allowed to proceed for a pre-determined period of time, after which the meter can activate a mechanism for transferring reacted liquid sample 32 from reaction chamber 14 to detection chamber 16, for example, by creating opening 40 in sealing sheet 28 or a similar opening in sealing sheet 26 which can allow the air trapped in detection chamber 16 to vent, allowing reacted liquid sample 32 to enter detection chamber 16 using capillary action. When reacted liquid sample 32 is transferred from reaction chamber 14 to detection chamber 16, liberated probe species 24 can transfer with reacted liquid sample 32 to detection chamber 16, but probe species 24 not liberated in the reaction can remain in reaction chamber 14. Upon reacted liquid sample 32 entering detection chamber 16, any reagent(s), for example, detection reagents, in detection chamber 16 can be dissolved and the detection reaction can proceed to generate one or more reaction products that can be detected by way of, for example, an optical method, an electrochemical method, or the like. In some embodiments, liberated probe species 24 can be detected directly. In FIGS. 1 and 2 electrochemical detection of the probe species is depicted, where conductive layers 38 and 36 on support layers 37 and 35 are shown. The portion of layers 38 and 36 in detection chamber 16 act as electrodes that can detect an electroactive species produced by reactions of liberated probe species 24. The signal resulting from such detection can then be processed by algorithms to derive a result in a desired format, such as a qualitative result as to whether the target analyte is present in the sample, a semi-quantitative result which can give an approximate range of the concentration or the target analyte in the sample, a quantitative estimate of the concentration of the target analyte in the liquid sample, or the like.

Some embodiments of the invention include a method of fabricating a biosensor disclosed herein. Exemplary methods of fabricating the biosensor can be found in, for example, PCT Patent Application Publication Nos. WO 2002/008763 entitled Immunosensor, WO 2007/096730 entitled FLUID TRANSFER MECHANISM, and WO 2010/004436 entitled ENHANCED IMMUNOASSAY SENSOR; and U.S. Patent Application Publication Nos. US 20030180814 entitled DIRECT IMMUNOSENSOR ASSAY and US 20060134713 entitled BIOSENSOR APPARATUS AND METHODS OF USE, and US 20060266644 entitled METHOD AND APPARATUS FOR ELECTROCHEMICAL ANALYSIS; and U.S. Pat. No. 8,192,599, entitled METHOD AND APPARATUS FOR ELECTROCHEMICAL ANALYSIS, each of which is hereby incorporated by reference.

Merely by way of example, with respect to the linker attached to (e.g., tethered to, supported on, or the like) a separate support (e.g., a bead, a magnetic bead, or the like), after the construct of the probe species linked to the linker has been attached to the support to generate a modified support, the modified support can be deposited in the first chamber (e.g., reaction chamber) of the strip during manufacture such that after manufacture it is in a dry form until the strip is used. The support can be substantially prevented from passing out of the first chamber (e.g., reaction chamber) to the second chamber (e.g., detection chamber) when a liquid sample is transferred from the first chamber (e.g., reaction chamber) to the second chamber (e.g., detection chamber) during a test. This can be achieved by, for example, physisorbing, chemisorbing the support to one or more internal surfaces of the first chamber (e.g., reaction chamber), or the like, by, for example, covalently linking the support to one or more internal surfaces of the first chamber (e.g., reaction chamber) or by applying a field that can retard the support from entering the second chamber (e.g., detection chamber) as the fluid is transferred. If magnetic beads are used then a magnetic field is a suitable field to apply such that it creates forces that retard the magnetic beads entering the second chamber (e.g., detection chamber).

Merely by way of example, the detection chamber can be constructed of materials that can be appropriate for the detection method or mechanism to be used. For example, if an optical method is used then the detection chamber can contain areas transparent to the stimulation (if present) and detection wavelengths to allow light to exit the chamber and be detected. Examples of suitable materials can include glass, polymers such as polystyrene, polycarbonate, and polyester, or the like. When an electrochemical detection method is used, the second chamber (e.g., detection chamber) can include one or more electrically conductive materials that can act as electrodes. At least two electrodes can be included, a working electrode and a counter or combined counter/reference electrode. A third reference electrode and other electrodes can also be included if desired. Suitable electrically conductive materials can include, for example, carbon, gold, palladium, platinum, iridium, or the like, or an alloy thereof, such as, for example, tin oxide, indium oxide and mixed indium oxide/tin oxide, or the like. Suitable methods for electrochemical detection can be found in, for example, PCT Patent Application Publication Nos. WO 2002/008763 entitled Immunosensor, WO 2007/096730 entitled FLUID TRANSFER MECHANISM, and WO 2010/004436 entitled ENHANCED IMMUNOASSAY SENSOR; and U.S. Patent Application Publication Nos. US 20030180814 entitled DIRECT IMMUNOSENSOR ASSAY and US 20060134713 entitled BIOSENSOR APPARATUS AND METHODS OF USE, and U.S. Patent Nos. 20060266644 entitled METHOD AND APPARATUS FOR ELECTROCHEMICAL ANALYSIS; and U.S. Pat. No. 8,192,599, entitled METHOD AND APPARATUS FOR ELECTROCHEMICAL ANALYSIS, each of which is hereby incorporated by reference. The electrically conductive materials can be supported on support layers to give them increased mechanical strength. These layers can be electrically conductive or electrically insulating. The electrodes can be electrically isolated from one another. If the working and counter electrodes are on the same support layer, they cannot be in direct contact or otherwise electrically connected with the support layer if the support layer is electrically conductive. In some embodiments, the support layer can be made of an electrically insulating material such as, for example, polymer, glass, ceramic, or the like. In some embodiments, polymers such as, for example, polyester, polyimide, or the like, which is inert and flexible, can be beneficial.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A biosensor for detecting a target analyte in a liquid sample, wherein the biosensor comprises at least a first chamber and a second chamber, wherein the first chamber and the second chamber are in fluid communication, wherein the first chamber comprises a probe species, wherein the probe species is retained in the first chamber by a linker, wherein the target analyte is capable of cleaving the linker to liberate the probe species into the liquid sample in the first chamber, wherein the biosensor is configured to move the liquid sample including the liberated probe species from the first chamber to the second chamber, and wherein the liberated probe species is detected in the second chamber via a detection mechanism.

2. The biosensor of claim 1, wherein the linker is attached to an internal surface of the first chamber.

3. The biosensor of claim 1, wherein the linker is attached to a separate support.

4. The biosensor of claim 3, wherein the separate support comprises a bead.

5. The biosensor of claim 4, wherein the bead is magnetic.

6.-8. (canceled)

9. The biosensor of claim 1, wherein the target analyte comprises an enzyme.

10. The biosensor of claim 9, wherein the target analyte comprises at least one enzyme selected from the group consisting of a chymotrypsin, a pepsin, a papain, an isopeptidase, a thrombin, a lactase, a maltase, a sucrase, an amylase, a pappalysin-2, a lysozyme, a protease, and a matrix metalloproteinase.

11.-14. (canceled)

15. The biosensor of claim 1, the probe species comprises an optically active molecule, an enzyme, and an electrically active molecule.

16. The biosensor of claim 1, wherein the liberated probe species is detected directly via the detection mechanism.

17. The biosensor of claim 1, wherein the liberated probe species comprises an enzyme that undergoes a detection reaction, wherein the detection reaction generates a reaction product, wherein the reaction product is detected via the detection mechanism.

18.-20. (canceled)

21. The biosensor of claim 1, wherein the detection mechanism comprises one mechanism selected from the group consisting of reflectance spectroscopy, transmission spectroscopy, fluorometry, turbidimetry, chemiluminescence microscopy, coulometry, an amperometry, and potentiometry.

22. The biosensor of claim 1, wherein the first chamber is a reaction chamber, and wherein the second chamber is a detection chamber.

23. The biosensor of claim 1 further comprising a filling chamber, wherein the filling chamber is in fluid communication with the first chamber.

24. The biosensor of claim 1, wherein the biosensor is configured to move the liquid sample via capillary action.

25. The biosensor of claim 1, wherein biosensor is configured to move the liquid sample from the first chamber to the second chamber upon activation.

26. The biosensor of claim 25, wherein the first chamber has a first height, wherein the second chamber has a second height, wherein the second height is smaller than the first height, and wherein the activation comprises opening a vent in the second chamber.

27. The biosensor of claim 26, wherein the vent is located at the distal end of the detection chamber.

28. The biosensor of claim 1, wherein the second chamber comprises two or more electrodes.

29. The biosensor of claim 28, wherein each of at least two of the two or more electrodes is electrically connected to a contact pad.

30. A system for detecting a target analyte in a liquid sample, wherein the system comprises a biosensor of claim 1 and a meter.

31.-50. (canceled)

51. A method of detecting a target analyte in a liquid sample using a biosensor, wherein the biosensor comprises a first chamber and a second chamber, wherein the first chamber comprises a probe species, wherein the probe species is retained in the first chamber via a linker, wherein the target analyte is capable of cleaving the linker to liberate the probe species, wherein the method comprises providing the liquid sample;
allowing the liquid sample to remain in the first chamber of the biosensor to generate a reacted liquid sample;
advancing the reacted liquid sample to the second chamber of the biosensor; and
measuring a detectable signal in the second chamber of the biosensor;
wherein the detectable signal indicates the presence and/or amount of the target analyte in the liquid sample.

52.-57. (canceled)
